SEQUENCE LISTING (8 sequences)

SEQ ID NO: 1 - DNA, 43 bp, Artificial
  Synthetic
tccctacacgacgctcttccgatctattgcctctgataagtac

SEQ ID NO: 2 - DNA, 52 bp, Artificial
  Synthetic
gtgactggagttcagacgtgtgctcttccgatctatgacagccccagcccat

SEQ ID NO: 3 - DNA, 45 bp, Artificial
  Synthetic
tccctacacgacgctcttccgatctagacaagcctcagttggatc

SEQ ID NO: 4 - DNA, 54 bp, Artificial
  Synthetic
gtgactggagttcagacgtgtgctcttccgatctatcccaaggctacatcatta

SEQ ID NO: 5 - DNA, 45 bp, Artificial
  Synthetic
tccctacacgacgctcttccgatctgggagtcatggaaactgatc

SEQ ID NO: 6 - DNA, 53 bp, Artificial
  Synthetic
gtgactggagttcagacgtgtgctcttccgatctttgatagtaacaaggcccc

SEQ ID NO: 7 - DNA, 45 bp, Artificial
  Synthetic
tccctacacgacgctcttccgatctattcttcttctgaaaggatc

SEQ ID NO: 8 - DNA, 54 bp, Artificial
  Synthetic
gtgactggagttcagacgtgtgctcttccgatctattttagcggaagactcaca

(19) United States
(12) Patent Application Publication
(10) Pub. No.: US 2019/0203203 A1
(43) Pub. Date: Jul. 4, 2019

(54) GENOME-WIDE IDENTIFICATION OF CHROMATIN INTERACTIONS

(71) Applicant: Ludwig Institute for Cancer Research Ltd, Zurich (CH)

(72) Inventors: Bing Ren, La Jolla, CA (US); Miao Yu, La Jolla, CA (US); Rongxin Fang, La Jolla, CA (US)

(21) Appl. No.: 16/330,002

(22) PCT Filed: Aug. 31, 2017
(86) PCT No.: PCT/US17/49549

§ 371 Date: Mar. 1, 2019

Related U.S. Application Data
(60) Provisional application No. 62/383,112, filed on Sep. 2, 2016.
(60) Provisional application No. 62/398,175, filed on Sep. 22, 2016.

(51) Int. Cl. C12N 15/10

(52) U.S. Cl. CPC C12N 15/1065

(57) ABSTRACT

Methods and kits for genome-wide identification of chromatin interactions in a cell are provided.

### CROSS REFERENCE TO RELATED APPLICATION

This application claims priority to U.S. Provisional Application No. 62/383,112 filed on Sep. 2, 2016 and U.S. Provisional Application No. 62/398,175 filed on Sep. 22, 2016. The contents of the applications are incorporated herein by reference in their entireties.

### STATEMENT REGARDING FEDERALLY FUNDED RESEARCH AND DEVELOPMENT

This invention was made with government support under grant numbers 1U54DK107977-01 and U54 HG006997 awarded by the National Institutes of Health. The United States government has certain rights to this invention.

### BACKGROUND OF THE INVENTION

Formation of long-range chromatin interactions is a crucial step in transcriptional activation of target genes by distal enhancers. Mapping of such structural features can help to define target genes for cis regulatory elements and annotate the function of non-coding sequence variants linked to human diseases (Gorkin, D. U., et al., Cell Stem Cell 14, 762-775 (2014), de Laat, W. & Duboule, D. Nature 502, 499-506 (2013), Sexton, T. & Cavalli, G. T. Cell 160, 1049-1059 (2015), and Babu, D. & Fullwood, M. J. Nucleus 6, 382-393 (2015)). Study of long-range chromatin interactions and their role in gene regulation has been facilitated by the development of chromatin conformation capture (3C)-based technologies (Dekker, J., et al., Nat. Rev. Genet. 14, 390-403 (2013) and Denker, A. & de Laat, W. Genes & development 30, 1357-1382 (2016)). Among the commonly used high-throughput 3C approaches are Hi-C and ChIA-PET (Lieberman, E. Science 326, 289-293 (2009) and Fullwood, M. J. et al., Nature 462, 58-64 (2009)). Global analysis of long-range chromatin interactions using Hi-C has been achieved at kilobase resolution, but requires billions of sequencing reads (Rao, S. S. P. et al., Cell 159, 1665-1680 (2014)). High-resolution analysis of long-range chromatin interactions at selected genomic regions can be attained cost-effectively through either chromatin analysis by paired-end tag sequencing (ChIA-PET), or targeted capture and sequencing of Hi-C libraries (Fullwood, M. J. et al., Nature 462, 58-64 (2009), Mifsud, B. et al., Nat. Genet. 47, 598-606 (2015), and Tang, Z. et al., Cell 163, 1611-1627 (2015)). Specifically, ChIA-PET has been successfully used to study long-range interactions associated with proteins of interest at high-resolution in many cell types and species (Li, G. et al., BMC Genomics 15 Suppl 12, S11 (2014)). However, the requirement for tens to hundreds of million cells as starting materials has limited its application.

### SUMMARY OF THE INVENTION

In certain embodiments, methods for genome-wide identification of chromatin interactions in cells are provided.

In certain embodiments, the method comprises providing a cell that contains a set of chromosomes having genomic DNA; incubating the cell or the nuclei thereof with a fixation agent to provide fixed cells comprising crosslinked DNA; performing proximity ligation of the genomic DNA of the fixed cells; isolating chromatin from the cells to provide a library; and sequencing the library. The proximity ligation can be an ex situ ligation or an in situ ligation.

In some embodiments, the cell is a eukaryotic cell. In some embodiments, the cell is a mammalian cell. In some embodiments, the cell is a human cell. In some embodiments, the fixation agent is formaldehyde, glutaraldehyde, formalin, or a mixture thereof. In some embodiments, the proximity ligation is an in situ proximity ligation. The in situ proximity ligation can be performed by permeabilizing the fixed cells, fragmenting the DNA by restriction enzyme digestion, followed by labeled nucleotide fill-in and proximity ligation. Restriction enzyme digestion may be carried out with one or more enzymes. The enzyme may be a 4-cutter or a 6-cutter. In one embodiment the enzyme is MboI. Labeled nucleotide fill-in may be performed by incubation with and DNA polymerase, for example Klenow, and dCTP, dGTP, dTTP, and dATP, one of which is labeled with a label. In one embodiment, the label is biotin. Proximity ligation may be performed by incubation with a ligase in a ligase buffer.

In some embodiments, chromatin is isolated by immunoprecipitation. In some embodiments, chromatin is isolated by lysing the nucleus of the cell, shearing the chromatin by sonication to provide a soluble chromatin fraction, and subjecting the soluble chromatin fraction to immunoprecipitation. In some embodiments, immunoprecipitation is performed with specific antibodies against either a DNA bound protein or histone modification. In some embodiments, after the step of isolating the chromatin, reverse-crosslinking is performed and labeled junctions are enriched before paired-end sequencing.

In some embodiments, kits for performing the methods of the invention are provided. The kits may contain one or more of a fixation agent, a restriction enzyme, one or more reagents for affinity tag filling in, one or more reagents for proximity ligation, one or more reagents for chromatin isolation, and one or more reagents for sequencing. Examples of reagents for chromatin isolation include reagents for immunoprecipitation and affinity tag pulling down as described herein.

### BRIEF DESCRIPTION OF THE DRAWINGS

FIGS. 1a, 1b, 1c, 1d, 1e, 1f, 1g, 1h, 1i and 1j illustrate chromatin interactions in mammalian cells determined by using a PLAC-seq method. (a) Overview of PLAC-seq workflow. Formaldehyde-fixed cells are permeabilized and digested with 4-bp cutter MboI, followed by biotin fill-in and in situ proximity ligation. Nuclei are then lysed and chromatins sheared by sonication. The soluble chromatin fraction is then subjected to immunoprecipitation with specific antibodies against either a DNA bound protein or histone modification. Finally, reverse-crosslinking is performed and biotin-labeled ligation junctions are enriched before paired-end sequencing. (b) Comparison of sequencing outputs from the Pol II PLAC-seq and ChIA-PET experiments. (c-d) Browser plots show examples of high-resolution long-range interactions revealed by H3K27Ac and Pol II PLAC-seq. c, promoter-promoter interactions; d, left panel, enhancer-enhancer interactions; d, right panel, promoter-enhancer interactions. (e) Box plots of raw reads count for ChIA-PET and PLAC-seq interactions. (f) Overlap between Pol II PLAC-seq and Pol II ChIA-PET interactions. (g) Sensitivity and accuracy of PLAC-seq and ChIA-PET interactions compared to in situ Hi-C identified interactions. (h) Overlap of interactions identified by H3K27ac, H3K4me3 PLAC-seq and in situ Hi-C. (i) Comparison of coverage of promoters and distal DHSs between PLAC-seq and ChIA-PET. (j) Comparison of 4C-seq, PLAC-seq, ChlA-PET anchored at Mreg promoter and a putative enhancer (1,2,3 highlight interactions not detected by ChIA-PET; 4C anchor points are marked by asterisk while PLAC-seq and ChIA-PET anchor regions are marked by black rectangle).

FIGS. 2a, 2b, 2c, and 2d illustrate identification of promoter and enhancer interactions in mESC. (a) PLAC-seq interactions are enriched at genomic regions associated with the corresponding histone modifications. (b) Overlap between H3K27ac and H3K4me3 PLAC-Enriched (PLACE) interactions. (c) Distribution of promoter-promoter, promoter-enhancer, enhancer-enhancer and other interactions for H3K27ac and H3K4me3 PLACE interactions. (d) Boxplot of expression of different groups of genes. H3K27ac PLACE interactions are associated with genes express significantly higher than other genes (Wilcoxon tests, P<2.2e-16).

FIGS. 3a, 3b, 3c, 3d, 3e, 3f, and 3g illustrate the validation of PLAC-seq. (a) Comparison of input material requirement of PLAC-seq and ChIA-PET. (b) Principal component analysis (PCA) of short-range reads in different PLAC-seq experiments highlights the reproducibility between biological replicates. (c) Box plots of Reads Per Kilobase per Million reads (RPKM) calculated using PLAC-seq short-range cis pairs (distance <1 kb) suggest that PLAC-seq signals are significantly enriched in ChIP-seq peaks compared to randomly chosen regions (***Wilcoxon tests, P<2.2e-16). (d) The signals of short-range reads (<1 kb) from PLAC-seq were similar to those of ChIP-seq. (e) Box plots of reads per million (RPM) at ChIP-enriched regions for PLAC-seq and in situ Hi-C. Only long-range (>10 kb) cis reads were considered (***Wilcoxon tests, P<2.2e16). (f) Scatter plots of pair-wise interaction frequency on chromosome 3. Left, PLAC-seq biological replicates were highly reproducible (R²=0.90); right, interaction intensity is skewed towards PLAC-seq for fragments with H3K27ac ChIP-seq peaks comparing to in situ Hi-C (R²=0.76). (Dots in the oval represent fragment pairs with at least one end bound by H3K27ac) (g) Example of long-range cis reads enrichment in H3K27ac, H3K4me and Pol II PLAC-seq compared to in situ Hi-C (visualized by Juicebox).

FIG. 4 shows scatter plots of interaction intensity between PLAC-seq biological replicates (left panels) and between PLAC-seq and in situ Hi-C (right panels) on chromosome 3. (Dots in the oval represent fragment pairs bound by corresponding ChIP-seq peaks).

FIGS. 5a and 5b illustrate PLAC-seq data by 4V-seq. (a) Long-range interactions identified by H3K27ac PLAC-seq are reproducible using different number of cells. (b) Comparison of 4C, PLAC-seq, ChIA-PET results on the selected locus. (4C anchor points are marked by asterisk while PLAC-seq and ChIA-PET anchor regions are marked by black rectangle; the right rectangle highlights chromatin interaction uniquely detected by ChIA-PET but not observed from 4C-seq).

### DETAILED DESCRIPTION OF THE INVENTION

This invention is based, at least in part, on an unexpected discovery that combining proximity ligation with chromatin immunoprecipitation and sequencing allows one to achieve genome-wide identification of chromatin interactions in a highly sensitive and cost-effective way. This approach exhibits superior sensitivity, accuracy and ease of operation. For example, application of the approach to eukaryotic cells improves mapping of enhancer-promoter interactions.

As noted above, the formation of long range chromatin interactions is a crucial step in transcriptional activation of target genes by distal enhancers. Mapping of these interactions helps to define target genes for cis regulatory elements and annotate the function of non-coding sequence variants linked to various physiological and pathological conditions. Conventional approaches for such mapping generally require a large number of cells and deep sequencing. For example, billions of sequencing reads are often needed to obtain satisfactory coverage. This is very costly and not sensitive or accurate.

Disclosed herein is a new method for genome-wide identification of chromatin interactions. This method, which is referred as Proximity Ligation Assisted ChIP-seq (PLAC-seq), takes advantages of proximity ligation-based chromatin interaction analysis and protein-specific DNA binding, and thereby achieves superior long range chromatin interaction mapping. As disclosed below, this method can generate more comprehensive and accurate interaction maps than ChIA-PET. The ease of experimental procedure, the low amount of cells required and the cost-effectiveness of this method greatly facilitate the mapping of long-range chromatin interactions in a much broader set of species, cell types and experimental settings than previous approaches.

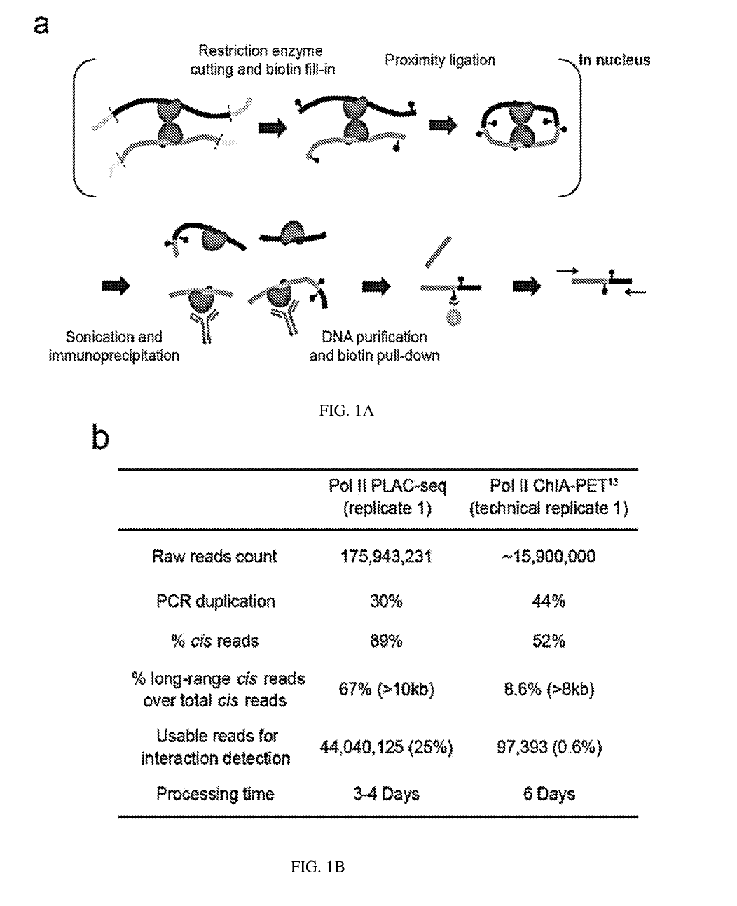

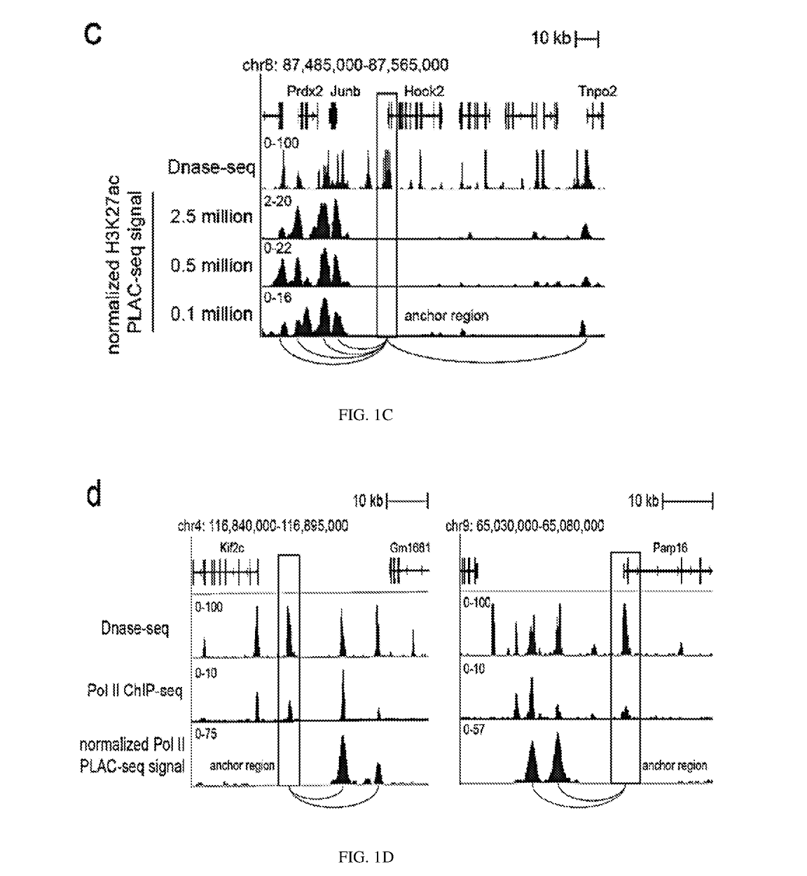

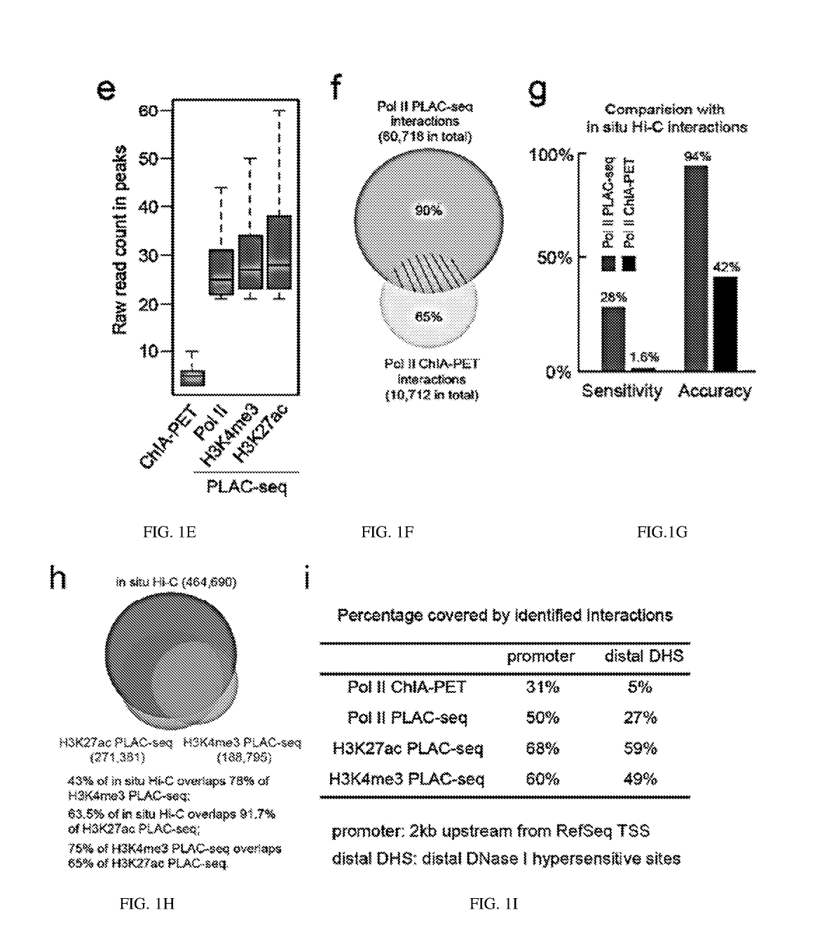

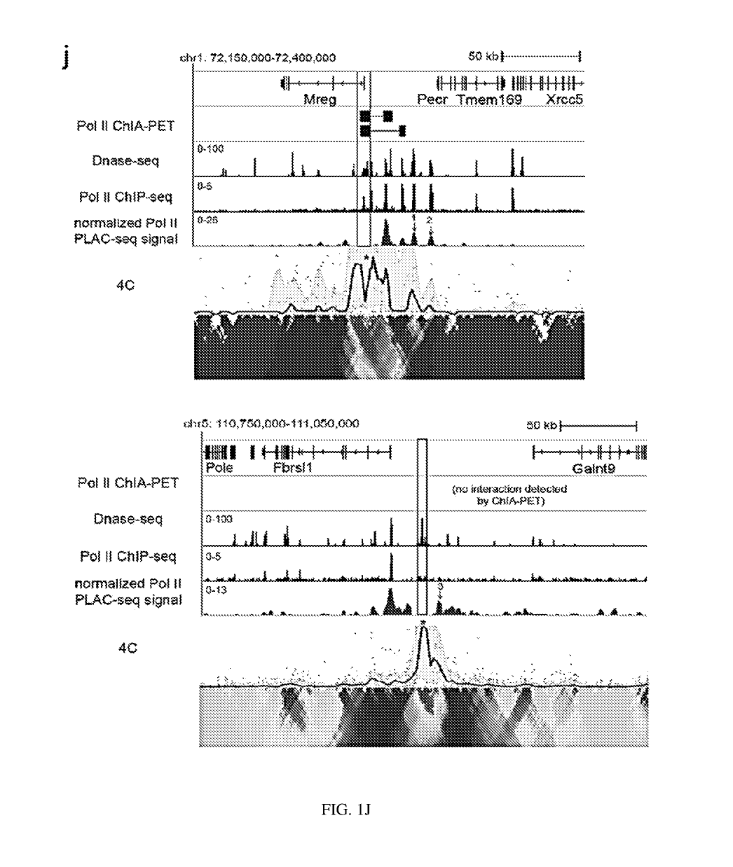

The method generally includes: providing a cell that contains a set of chromosomes having genomic DNA; incubating the cell or the nuclei thereof with a fixation agent to provide a fixed cell comprising a complex having genomic DNA crosslinked with a protein; performing in situ proximity ligation of the genomic DNA of the fixed cell to form proximally-ligated genomic DNA; isolating the complex from the cell to provide a DNA library; and sequencing the DNA library. Part of the workflow is shown in FIG. 1A. Some of the steps are further described below.

Crosslinking

The method disclosed herein includes an in vitro technique to fix and capture associations among distant regions of a genome as needed for long-range linkage and phasing.

The technique utilizes fixation of chromatin in live cells to cement spatial relationships in the nucleus. With this fixation, subsequent processing of the products allows one to recover a matrix of proximate associations among genomic regions. With further analysis these associations can be used to produce a three-dimensional geometric map of the chromosomes as they are physically arranged in live nuclei. Such techniques describe the discrete spatial organization of chromosomes in live cells, and provide an accurate view of the functional interactions among chromosomal loci. One issue that limited conventional functional studies is the presence of nonspecific interactions, associations present in the data that are attributable to nothing more than chromosomal proximity. In the disclosure, these nonspecific interactions are minimized by the method disclosed herein so as to provide valuable information for assembly in a more sensitive, accurate, and cost effective way.

More specifically, cross-links can be created between genome regions and proteins that are in close physical proximity. Crosslinking of proteins (such as histones) to the DNA molecule, e.g., genomic DNA, within chromatin can be accomplished according to a suitable method described herein or known in the art. In some cases, two or more nucleotide sequences can be cross-linked via proteins bound to one or more nucleotide sequences. Crosslinking of polynucleotide segments may also be performed utilizing many approaches, such as chemical or physical (e.g., optical) crosslinking. Suitable chemical crosslinking agents include, but are not limited to, formaldehyde, glutaraldehyde, formalin, and psoralen (Solomon et al., Proc. NatL. Acad. Sci. USA 82:6470-6474, 1985; Solomon et al., Cell 53:937-947, 1988). For example, cross-linking can be performed by adding 2% formaldehyde to a mixture comprising the DNA molecule and chromatin proteins. Other examples of agents that can be used to crosslink DNA include, but are not limited to, mitomycin C, nitrogen mustard, melphalan, 1,3-butadiene diepoxide, cis diaminedichloroplatinum (II) and cyclophosphamide. Suitably, the cross-linking agent will form cross-links that bridge relatively short distances-such as about 2 Å-thereby selecting intimate interactions that can be reversed. Another approach is to expose the chromatin to physical (e.g., optical) crosslinking, such as ultraviolet irradiation (Gilmour et al., Proc. Nat'l. Acad. Sci. USA 81:4275-4279, 1984).

Genomic DNA Fragmenting and Affinity Tag Filling in

The method described herein involves fragmenting genomic DNA prior to proximity-ligation of chromatin. Many methods for DNA fragmenting are known in the art. Thus, fragmentation can be accomplished using established methods for fragmenting chromatin, including, for example, sonication, shearing and/or the use of enzymes, such as restriction enzymes.

In some embodiments, a restriction enzyme digestion is used. As most of the sequencing reads are distributed near (˜500 bp) the restriction enzyme cut-site, the choice of enzyme used can impact the results. To maximize identification of chromatin interactions, one can use multiple enzymes for chromatin digestion. To this end, any single 6-base cutting restriction enzyme can generate proximity-ligation data that covers 5-10% of the genome, but by using multiple such enzymes in the same experiment, one can cover >80% of the genome. In addition, a 4-base cutter enzyme or a set of 4-base cutters can be used instead of 6-base cutting enzymes to further maximize the coverage of the genome.

The PLAC-seq procedure disclosed herein can be performed using any number of restriction enzymes provided that they generate sufficient libraries. The issue of enzyme choice does have an effect in terms of the number of bases that are covered and mapped. For instance, 6-base cutting enzymes cut every ˜4 kb in the genome, and therefore a relative minority of polymorphisms that could be phased falls close enough to cut sites to be phased. In contrast, 4-base cutting enzymes cut much more frequently, on the order of every 250 bp (on average). In this regard, a much larger percentage of polymorphisms will fall close to enzyme cut sites and therefore have the potential to be phased. This is implicated for phasing of rare variants.

Generally, utilizing a 4-base cutting enzyme or a mixture of different enzymes led to greater coverage with less sequencing read depth. Here, while PLAC-seq may be successfully performed using one restriction enzyme, PLAC-seq using multiple enzymes can generate more uniform distribution of data and consequently higher-resolution map. Restriction enzyme can have a restriction site of 1, 2, 3, 4, 5, 6, 7, or 8 bases long. Examples of restriction enzymes include but are not limited to Aatll, Acc65I, Accl, Acil, Acll Acul, Afel, Aflll, Afllll, Agel, Ahdl, Alel, Alul, Alwl, AlwNI, Apal, ApaLI, ApeKI, Apol, Ascl, Asel, AsiSI, Aval, Avail, Avrll, BaeGI, Bael, BamHI, Banl, Banll, Bbsl, BbvCI, Bbvl, Bed, BceAI, Bcgl, BciVI, Bell, Bfal, BfuAI, BfuCI, Bgll, Bgill, Blpl, BmgBI, Bmrl, Bmtl, Bpml, BpulOI, BpuEI, BsaAI, BsaBI, BsaHI, Bsal, BsaJI, BsaWI, BsaXI, BscRI, BscYI, Bsgl, BsiEI, BsiHKAI, Bsi I, BslI, BsmAI, Bs BI, Bs FI, Bsml, BsoBI, Bspl286I, BspCNI, BspDI, BspEI, BspHI, BspMI, BspQI, BsrBI, BsrDI, BsrFI, BsrGI, Bsrl, BssHII, BssKI, BssSI, BstAPI, BstBI, BstEII, BstNI, BstUI, BstXI, BstYI, BstZ17I, Bsu36I, Btgl, BtgZI, BtsCI, Btsl, CacSI, Clal, CspCI, CviAII, CviKI-1, CviQI, Ddcl, DpnI, DpnII, Dral, DralIL Drdl, Eacl, Eagl, Earl, Ecil, Eco53kI, Eco I, EcoO109I, EcoP15I, EcoRI, EcoRV, Fatl, Fad, Fnu4HI, Fokl, Fsel, Fspl, Haell, Haelll, figal, Hhal, Hindi, HindIII, Hinfl, HinPlI, Hpal, Hpall, Hphl, Hpyl66II, Hpyl88I, Hpyl88III, Hpy99I, HpyAV, HpyCH4III, HpyCH4IV, HpyCH4V, Kasl, Kpnl, Mbol, MbollI, Mfel, Mlul, Mlyl, Mmel, Mnll, Mscl, Mse, MslI, MspAlI, Mspl, Mwol, Nael, Narl, Nb.BbvCI, Nb.Bsml, Nb.BsrDI, Nb.BtsI, Neil, col, Ndel, NgoMIV, Nhel, Nla 11, NlalV, NmeAIII, Notl, Nrul, Nsil, Nspl, Nt.AlwI, Nt.BbvCI, Nt.BsmAI, Nt.BspQI, Nt.BstNBI, Nt.CviPII, Pad, PaeR7I, Pcil, PflFI, PflMI, Phol, Ple, Pmel, Pmll, PpuMI, PshAI, Psil, PspGI, PspOMI, PspX, Pstl, Pvul, Pvul I, P.sal, RsrII, Sad, SacII, Sail, Sapl, Sau3AI, Sau96I, Sbfl, Seal, ScrFI, SexAI, SfaNI, Sfcl, Sfil, Sfol, SgrAI, Smal, Smll, SnaBI, Spel, Sphl, Sspl, Stul, StyD4I, Styl, Sv/al, T, Taqal, Tfil, Tlil, Tsel, Tsp45I, Tsp509I, TspMI, TspRI, Tthllll, Xbal, Xcml, Xhol, Xmal, Xmnl, and Zral. The resulting fragments can vary in size. The resulting fragments may also comprise single-stranded overhands at the 5′ or 3′ end.

These single-stranded overhands at the 5′ or 3′ end can be filled by nucleotides labelled with one or more affinity tags. Examples of the affinity tag include a biotin molecule, a hapten, glutathione-S-transferase, and maltose binding protein. Techniques for capture tag filling-in are known in the art.

Proximity Ligation

In the workflow shown in FIG. 1a, a proximity-ligation based method is used for DNA sequencing library preparation, followed by high throughput DNA sequencing. The proximity ligation may occur (1) within intact cells (i.e. in situ proximity ligation, e.g. similar to the steps described in Rao, S. S. P. et al., Cell 159, 1665-1680 (2014)) or (2) using lysed cells, lysed nuclei or cellular components (i.e. ex situ proximity ligation, e.g. similar to the steps described in Lieberman-Aiden et al. Science 326, 289-93 (2009), Selvaraj et al. Nat Biotechnol 31, 1111-8 (2013), or WO2015010051, the contents of all of which are incorporated herein by reference). More specifically, cells may be cross-linked with a crosslinking agent to preserve protein-protein and DNA-protein interactions. This step may be carried out at room temperature for 10-30 minutes with 1-2% of formaldehyde. The cells may then be harvested by centrifugation and may be stored at −80° C. The cells may be lysed in a hypotonic nuclear lysis buffer, and then washed with a 1× concentration of buffer for the restriction enzyme of choice (e.g., from New England Biolabs). The cells may be digested for 1 hour to overnight with 25 U to 400 U of enzyme, depending upon the enzyme used. Four-base cutting enzymes benefit from short digestions with less amount of enzyme (e.g., 1 hour with 25 U), whereas six-base cutting enzymes can use longer digestions with larger amounts of enzyme. The ends of DNA may be repaired with Klenow polymerase in the presence of dNTPs, one of which (e.g., dATP) may be covalently linked to an affinity tag, such as biotin. The sample may then be ligated in the presence of T4 DNA ligase for 4 hours.

As shown in FIG. 1a, the proximity-ligation generates complexes having DNA-binding protein and proximity-ligated DNA pairs. These complexes may be further sheared and isolated by e.g., immunoprecipitation, as described below.

Shearing

Before isolating, the complexes may be further processed. As mentioned above, many methods for shearing DNA are known in the art and can be used here. Shearing can be accomplished using established methods for fragmenting chromatin, including, for example, sonication and/or the use of restriction enzymes. In some embodiments, using sonication techniques, fragments of about 100 to 5000 nucleotides can be obtained.

Immunoprecipitation

Various techniques can be used to isolate the complexes mentioned above. In one embodiment, immunoprecipitation may be used. This isolation technique allows precipitating a protein antigen (such as a DNA-binding protein), as well as other molecules complexed with it (such as genomic DNA), out of solution using an antibody that specifically binds to that particular protein antigen. This process can be used to isolate and concentrate a particular protein from a sample containing many thousands of different proteins. Immunoprecipitation can be carried out with the antibody being coupled to a solid substrate at some point in the procedure.

As disclosed herein, useful protein antigens in general are DNA-binding proteins (including transcription factors, histones, polymerases, and nucleases) or others associated with such DNA-binding proteins. As disclosed above, the proteins are cross-linked to the DNA that they are binding to. By using an antibody that is specific to such a DNA-binding protein, one can immunoprecipitate the protein-DNA complex out of cellular lysates. The crosslinking can be accomplished by applying a fixation agent, e.g., formaldehyde, to the cells (or tissue), although it is sometimes advantageous to use a more defined and consistent crosslinker known in the art (such as Di-tert-butyl peroxide or DTBP). Following crosslinking, the cells may be lysed and the DNA may be broken into pieces in the manner described above. As a result of the immunoprecipitation, protein-DNA complexes are purified and the purified protein-DNA complexes can be heated to reverse the formaldehyde cross-linking of the protein and DNA complexes, allowing the DNA to be separated from the proteins.

The identity and quantity of the DNA fragments isolated can then be determined by various techniques, such as cloning, PCR, hybridization, sequencing, and DNA microarray (e.g., ChIP-on-chip or ChIP-chip).

Various DNA-binding proteins can be targets of the method disclosed herein. Examples of the DNA-binding proteins are described below. One potential technical hurdle with immunoprecipitation is the difficulty in generating an antibody that specifically targets a protein of interest. To get around this obstacle, one can engineer one or more tags onto either the C- or N-terminal end of the protein of interest to make an epitope-tagged recombinant protein. Such an epitope-tagged recombinant protein can be expressed in a cell of interest and then subject to the PLAC-seq disclosed herein. The advantage of epitope-tagging is that the same tag can be used time and again on many different proteins and the researcher can use the same antibody each time. Examples of tags in use are the Green Fluorescent Protein (GFP) tag, Glutathione-S-transferase (GST) tag, the HA tag, 6×His, and the FLAG-tag.

Affinity Tag Pull Down and Library Construction

The next step in the protocol is to capture and separate genomic DNA that has been immunoprecipitated for library construction. This can be performed via pull down of the affinity tags (e.g., biotin, a hapten, glutathione-S-transferase, or maltose binding protein). For example, the separating step can include contacting the immunoprecipitated mixture with an agent that binds to the affinity tag. Examples of the agent include an avidin molecule, or an antibody that binds to the hapten or an antigen-binding fragment thereof. In some embodiments, the agent can be attached to a support, such as a microarray. In that case, the support can include a planar support having one or more substrate materials selected from glass, silicas, metals, teflons, and polymeric materials. Alternatively, the support can include a mixture of beads, each bead having one or more affinity tag capture agent bound thereto and the mixture of beads can include one or more substrate materials selected from nitrocellulose, glass, silicas, teflons, metals, and polymeric materials. In some embodiments, the affinity tag pull down can be carried out in the manner described in Lieberman-Aiden, et al. Science 326, 289-93 (2009), Nat Biotechnol 31, 1111-8 (2013) and WO2015010051, the contents of which are incorporated herein by reference.

Adaptors (e.g., Illumina Tru-Seq adaptor) can then be ligated to the DNA. The sample can then amplified by PCR to obtain sufficient material. The PCR amplified libraries can be further purified. To maximize the PLAC-seq library complexity, the minimal number of PCR cycles for library amplification can be determined by qPCR against known standards to determine the number of cycles necessary to obtain enough material to sequence. The library can then be sequenced on, e.g., the Illumina sequencing platform.

Sequencing

Various suitable sequencing methods described herein or known in the art can be used to obtain sequence information from nucleic acid molecules within a sample. Sequencing can be accomplished through classic Sanger sequencing, massively parallel sequencing, next generation sequencing, polony sequencing, 454 pyrosequencing, Illumina sequencing, SOLEXA sequencing, SOLiD sequencing, ion semiconductor sequencing, DNA nanoball sequencing, heliscope single molecule sequencing, single molecule real time sequencing, nanopore DNA sequencing, tunneling currents DNA sequencing, sequencing by hybridization, sequencing with mass spectrometry, microfluidic Sanger sequencing, microscopy-based sequencing, RNA polymerase sequencing, in vitro virus high-throughput sequencing, Maxam-Gibler sequencing, single-end sequencing, paired-end sequencing, deep sequencing, ultradeep sequencing.

Reads from the sequencing may then be processed using bioinformatics pipelines to map long-range and/or genome wide chromatin interactions. For example, paired-end sequences can be first mapped using BWA-MEM (Li H. Aligning sequence reads, clone sequences and assembly contigs with BWA-MEM. arXiv:1303.3997v2 (2013)) to the reference genome (mm9) in single-end mode with default setting for each of the two ends separately. Next, independently mapped ends may be paired up and pairs are only kept if each of both ends are uniquely mapped (MQAL>10). For intrachromosomal analysis in this study, interchromosomal pairs may be discarded. Next, read pairs may be further discarded if either end is mapped more than 500 bp apart away from the closest restricting site (e.g., MboI site). Read pairs may next be sorted based on genomic coordinates followed by PCR duplicate removal using MarkDuplicates in Picard tools. Next, the mapped pairs may be partitioned into “long-range” and “short-range” if the insert size is greater than the given distance of the default threshold 10 kb or smaller than 1 kb, respectively.

DNA-Binding Proteins

The method disclosed herein may involve isolating DNA-binding proteins. Examples of DNA-binding proteins include transcription factors (TFs) which modulate the process of transcription, various polymerases, ligases, nucleases which cleave DNA molecules, and chromatin-associated proteins such as the histones, the high mobility group (HMG) proteins, methylases, helicases and single-stranded binding proteins, topoisomerases, recombinase, and the chromodomain proteins, which are involved in chromosome packaging and transcription in the cell nucleus. See, e.g., US20020186569.

DNA-binding proteins may include such domains as the zinc finger, the helix-loop-helix, the helix-turn-helix, and the leucine zipper that facilitate binding to nucleic acid. There are also more unusual examples such as transcription activator like effectors. Various DNA-binding proteins can be used to practice the method disclosed herein to identify and analyze chromatin interactions involving these DNA-binding proteins in connection with related biological events, such as gene expression regulation, transcription, DNA duplication, repairing, and epigenetics such as imprinting.

While some proteins bind to DNA in a non-sequence specific manner, many proteins bind to specific DNA sequences. The most studied of these are transcription factors, which regulate transcription of genes. Each transcription factor binds to one specific set of DNA sequences and activates or inhibits the transcription of genes that have these sequences near their promoters. The transcription factors do this in two ways. Firstly, they can bind the RNA polymerase responsible for transcription, either directly or through other mediator proteins; this locates the polymerase at the promoter and allows it to begin transcription. Alternatively, transcription factors can bind enzymes that modify the histones at the promoter. This alters the accessibility of the DNA template to the polymerase. DNA targets occur throughout an organism's genome. Changes in the activity of one type of transcription factor can affect thousands of genes. Thus, these transcription factors are often the targets of the signal transduction processes that control responses to environmental changes or cellular differentiation and development. Accordingly, the method disclosed herein can be used to study and evaluate a transcription factor in these responses at a genome wide scale.

Transcription factors that can be targeted include general transcription factors, which are involved in the formation of a preinitiation complex, such as TFIIA, TFIIB, TFIID, TFIIE, TFIIF, and TFIIH. They are ubiquitous and interact with the core promoter region surrounding the transcription start site(s) of all class II genes. Additional examples include constitutively active transcription factors (e.g., Sp 1, NF1, CCAAT), conditionally active transcription factors, developmental- or cell-specific transcription factors (e.g., GATA, HNF, PIT-1, MyoD, MyfS, Hox, and Winged Helix), signal-dependent transcription factors which require external signal for activation. The signal can be extracellular ligand-dependent (i.e., endocrine or paracrine, such as nuclear receptors), intracellular ligand-dependent (i.e., autocrine, such as SREBP, p53, orphan nuclear receptors), or cell membrane receptor-dependent (e.g., those involving second messenger signaling cascades resulting in the phosphorylation of transcription factors, such as CREB, AP-1, Mef2, STAT, R-SMAD, NF-icB, Notch, TUBBY, and NFAT). These transcription factors can be those of various super classes including those having basic domains (e.g., leucine zipper factors, helix-loop-helix factors, helix-loop-helix/leucine zipper factors, NF-1 family, RF-X family, and bHSH), Zinc-coordinating DNA-binding domains (e.g., Cys4 zinc finger of nuclear receptor type, diverse Cys4 zinc fingers, Cys2His2 zinc finger domain, Cys6 cysteine-zinc cluster, and Zinc fingers of alternating composition), helix-turn-helix (e.g., homeo domain, paired box, fork head/winged helix, heat shock factors, tryptophan clusters, and transcriptional enhancer factor) domain), or beta-scaffold factors with minor groove contacts (e.g., RHR, STAT, p53 class, MADS box, beta-Barrel alpha-helix transcription factors, TATA binding proteins, HMG-box, heteromeric CCAAT factors, grainyhead, cold-shock domain factors, and Runt), and others (e.g., copper fist proteins, HMGI(Y) (HMGA1), pocket domain, E1A-like factors, and AP2/EREBP-related factors).

Kits

The present disclosure further provides kits comprising one or more components for performing the method disclosed herein. The kits can be used for any application apparent to those of skill in the art, including those described above. The kits can comprise, for example, a plurality of association molecules, affinity tags, a fixative agent, a restriction endonuclease, a ligase, and/or a combination thereof. In some cases, the association molecules can be proteins including, for example, DNA binding proteins such as histones or transcription factors. In some cases, the fixative agent can be formaldehyde or any other DNA crosslinking agent. In some cases, the kit can further comprise a plurality of beads. The beads can be paramagnetic and/or may be coated with a capturing agent. For example, the beads can be coated with streptavidin and/or an antibody. In some cases, the kit can comprise adaptor oligonucleotides and/or sequencing primers. Further, the kit can comprise a device capable of amplifying the read-pairs using the adaptor oligonucleotides and/or sequencing primers. In some cases, the kit can also comprise other reagents including but not limited to lysis buffers, ligation reagents (e.g., dNTPs, polymerase, polynucleotide kinase, and/or ligase buffer, etc.), and PCR reagents (e.g., dNTPs, polymerase, and/or PCR buffer, etc.). The kit can also include instructions for using the components of the kit and/or for generating the read-pairs.

The kit may be in a container. The kit may also have containers for biological samples. In an exemplary case, the kit may be used for obtaining a sample from an organism. For example, the kit may comprise a container, a means for obtaining a sample, reagents for storing the sample, and instructions for use. In some cases, obtaining a sample from an organism may include extracting at least one nucleic acid from the sample obtained from an organism. For example, the kit may contain at least one buffer, reagent, container and sample transfer device for extracting at least one nucleic acid. In some cases, the kit may contain a material for analyzing at least one nucleic acid in a sample. For example, the material may include at least one control and reagent. The kit may contain polynucleotide cleavage agents (e.g., DNaseI, etc.) as well as buffers and reagents associated with carrying out polynucleotide cleavage reactions. In another exemplary case, the kit may contain materials for the identification of nucleic acids. For example, the kit may include reagents for performing at least one of the methods and compositions described herein. For example, the reagents may include a computer program for analyzing the data generated by the identification of nucleic acids. In some cases, the kit may further comprise software or a license to obtain and use software for analysis of the data provided using the methods and compositions described herein. In another exemplary case, the kit may contain a reagent that may be used to store and/or transport the biological sample to a testing facility.

Uses and Applications

The methods and kits described herein may be used to determine the pattern of proteins binding at sites within a nucleic acid. The methods and kits may further be used to correlate the protein-binding pattern to expression of genes within a nucleic acid sample or across multiple samples of nucleic acids. The methods and kits may be used to construct a regulatory network within a nucleic acid sample or across multiple samples of nucleic acids. Other examples for the uses include identification of functional variants/mutations in DNA-binding sites and/or regulatory DNA, identification of a transcript origination site, mapping of transcription factor networks in multiple cell types or multiple organisms, generating transcription factor networks, network analysis for cell-type-specific or cell-stage-specific behaviors of transcription factors, transcription factors and chromatin accessibility and function, promoter/enhancer chromatin signatures, disease- and trait-associated variants in regulatory DNA, disease-associated variants and transcriptional regulatory pathways, identification of diseased cells, and related screening assays.

The methods and kits may be used to determine the state of development, pluripotency, differentiation and/or immortalization of a nucleic acid sample; establish the temporal state of a nucleic acid sample; identify the physiologic and/or pathologic condition of the nucleic acid sample.

In one example, the methods and kits can be used for evaluating or predicting gene activation, transcription initiation, protein binding patterns, protein binding sites and chromatin structure. In some cases, the methods and kits can be used to detect temporal information about gene expression (e.g., past, future or present gene expression or activity). For example, the information may describe a gene activation event that occurred in the past. In some cases, the information may describe a gene activation event in the present. In some cases, the information may predict gene activation. The methods and kits described herein may be used to describe a physiologic state or a pathologic state. In some cases, the pathologic state may include the diagnosis and/or prognosis of a disease.

Using the methods disclosed herein, a large number (e.g., 10, 10², 10³, 10⁴, 10⁵, 10⁶, or 10⁷) of sites where proteins (e.g., transcription factors) bind a nucleic acid (e.g., genomic DNA) can be identified. In some cases, the binding of a transcription factor to a nucleic acid is within a regulatory region. These events may represent differential binding of a plurality of transcription factors to numerous distinct elements. In some cases, the number of distinct elements engaged or bound by transcription factors is greater than 10, 50, 500, 1000, 2500, 5000, 7500, 10000, 25000, 50000, or 100000. The distinct elements can be short sequence elements within a longer nucleic acid sequence. Differential binding of transcription factors to sequence elements can comprise a genomic sequence compartment that may encode a repertoire of conserved recognition sequences for DNA-binding proteins. The genomic sequence compartment may include sites previously known as well as novel sites that may have not yet been identified until use of the methods described herein. In some cases, the methods may be used to determine a cis-regulatory lexicon which may contain elements with evolutionary, structural and functional profiles.

In some cases, genetic variants that may affect allelic chromatin states may be identified. In some cases, the genetic variants may alter binding of proteins to the DNA sequence. In some cases, the genetic variants may be located in binding sites that may not be subject to modifications (e.g., DNA methylation).

The methods and kits can also be used to identify binding proteins (e.g., DNA-binding proteins) which recognize novel nucleic acid (e.g., DNA) sequences. The identification of binding proteins and recognition sequences can be performed either in vivo or in vitro. In some cases, the identification of binding proteins and recognition sequences may be performed in a sample taken from a single organism. In some cases, the identification of binding proteins and recognition sequences may be performed in a sample taken from a different organism. In some cases, the identification of binding proteins and recognition sequences may be analyzed across samples taken from at least one organism. For example, the analysis may determine that the identification of binding proteins and recognition sequences may have evolutionary functional signatures.

The methods can be used to identify novel regulatory factor recognition motifs. In some cases, the novel regulatory factor recognition motifs may be conserved in sequence and/or function across multiple genes, cell and/or tissue types within one species. In some cases, the recognition motifs may be conserved in sequence and/or function across multiple genes, cell and/or tissue types across a plurality of species. In some cases, the novel regulatory factor recognition motifs may not be conserved in sequence and/or function across multiple genes, cell and/or tissue types within one species. In some cases, the novel regulatory factor recognition motifs may not be conserved in sequence and/or function across multiple genes, cell and/or tissue types across a plurality of species. The novel regulatory factor recognition motifs may have cell-selective patterns of occupancy by one, or more than one, unique binding protein. The novel regulatory factor recognition motifs may not have cell-selective patterns of occupancy by one, or more than one, unique binding protein. In some cases, the novel regulatory factor recognition motifs may be arranged in a table, for example, a motif table.

Maps of long-range chromatin interactions (such as the PLACE interactions disclosed herein) may be assembled to depict a regulatory network (e.g., transcription factor network). Such maps of regulatory networks may provide a description of the circuitry, dynamics, and/or organizing principles of a regulatory network. For example, the maps may be generated from a library of polynucleotide fragments which, in some cases, may contain chromatin interaction sites. In some cases, the maps may include chromatin interactions across the entire genome. For example, the maps may be generated by aligning at least one library of polynucleotide fragments with at least one different library of polynucleotide fragments. In some cases, the polynucleotide fragment may be sequenced. In some cases, the aligning may be aligning the sequence of at least one polynucleotide with the sequence of at least one different polynucleotide. In some cases, the aligning may not include sequencing of at least one polynucleotide fragment. For example, the aligned libraries may include information that can be analyzed to determining a regulatory network. In some cases, the regulatory network can illustrate connections between hundreds of sequence-specific TFs. In some cases, the regulatory network can be used to analyze the dynamics of these connections across a plurality of cell and tissue types.

The cell and tissue samples may include several classes of cell types. Samples can include any biological material which may contain nucleic acid. Samples may originate from a variety of sources. In some cases, the sources may be humans, non-human mammals, mammals, animals, rodents, amphibians, fish, reptiles, microbes, bacteria, plants, fungus, yeast and/or viruses. Examples include cultured primary cells with limited proliferative potential, cultured immortalized, malignancy-derived or pluripotent cell lines, terminally differentiated cells, self-renewing cells, primary hematopoietic cells, purified differentiated hematopoietic cells, cells infected with a pathogen (e.g., virus) and/or a variety of multipotent progenitor and pluripotent cells or stem cells. In some cases, cell and tissue samples can be of post-conception fetal tissue samples.

Nucleic acid samples provided in this disclosure can be derived from an organism. To that end, an entire organism or a portion of it may be used. A portion of an organism may include an organ, a piece of tissue comprising multiple tissues, a piece of tissue comprising a single tissue, a plurality of cells of mixed tissue sources, a plurality of cells of a single tissue source, a single cell of a single tissue source, cell-free nucleic acid from a plurality of cells of mixed tissue source, cell-free nucleic acid from a plurality of cells of a single tissue source and cell-free nucleic acid from a single cell of a single tissue source and/or body fluids. In some cases, the portion of an organism is a compartment such as mitochondrion, nucleus, or other compartment described herein. A tissue can be derived from any of the germ layers, such as neural crest, endoderm, ectoderm and/or mesoderm. In some cases, the organ may contain a neoplasm such as a tumor. In some cases, the tumor may be cancer.

The sample may include cell cultures, tissue sections, frozen sections, biopsy samples and autopsy samples. The sample may be obtained for histologic purposes. The sample can be a clinical sample, an environmental sample or a research sample. Clinical samples can include nasopharyngeal wash, blood, plasma, cell-free plasma, buffy coat, saliva, urine, stool, sputum, mucous, wound swab, tissue biopsy, milk, a fluid aspirate, a swab (e.g., a nasopharyngeal swab), and/or tissue, among others. Environmental samples can include water, soil, aerosol, and/or air, among others. Samples can be collected for diagnostic purposes or for monitoring purposes (e.g., to monitor the course of a disease or disorder). For example, samples of polynucleotides may be collected or obtained from a subject having a disease or disorder, at risk of having a disease or disorder, or suspected of having a disease or disorder.

The methods can be applied to samples containing nucleic acid (e.g., genomic DNA) taken from multiple sources. The source may be a cell in a stage of cell behavior or stage. Examples of cell behavior include cell cycle, mitosis, meiosis, proliferation, differentiation, apoptosis, necrosis, senescence, non-dividing, quiescence, hyperplasia, neoplasia and/or pluripotency. In some cases, the cell may be in a phase or state of cellular maturity or aging. In some cases, the phase or state of cellular maturity may include a phase or state during the process of differentiation from a stem cell into a terminal cell type.

The PLAC-seq approach disclosed herein may be used to obtain respective PLACE (PLAC-Enriched) interaction for each cell behavior or stage or source. Each such interaction represents a gene regulation signature or profile specific for each cell behavior or stage or sources, and can be used for clinical purposes.

The methods and kits described herein can be used to screen at least one agent from a library of agents to identify an agent that may elicit a particular effect on the gene regulation signature or profile. The agent may be a drug, a chemical, a compound, a small molecule, a biosimilar, a pharmacomimetic, a sugar, a protein, a polypeptide, a polynucleotide, an RNA (e.g., siRNA), or a genetic therapeutic. The target may be an organism, an organ, a tissue, a cell, an organelle of a cell, a part of an organelle of a cell, chromatin, a protein, nucleic acid (e.g., genomic DNA) or a nucleic acid. The screen may include high-throughput screening and/or array screening, which may be combined with the methods and compositions described herein.

### Definitions

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

The term “about” generally refers to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

The term “biological sample” refers to a sample obtained from an organism (e.g., patient) or from components (e.g., cells) of an organism. The sample may be of any biological tissue, cell(s) or fluid. The sample may be a “clinical sample” which is a sample derived from a subject, such as a human patient. Such samples include, but are not limited to, saliva, sputum, blood, blood cells (e.g., white cells), amniotic fluid, plasma, semen, bone marrow, and tissue or fine needle biopsy samples, urine, peritoneal fluid, and pleural fluid, or cells therefrom. Biological samples may also include sections of tissues such as frozen sections taken for histological purposes. A biological sample may also include a substantially purified or isolated protein, membrane preparation, or cell culture.

A “nucleic acid” refers to a DNA molecule (e.g., a genomic DNA), an RNA molecule (e.g., an mRNA), or a DNA or RNA analog. A DNA or RNA analog can be synthesized from nucleotide analogs. The nucleic acid molecule can be single-stranded or double-stranded, but preferably is double-stranded DNA.

The term “labeled nucleotide” or “labeled base” refers to a nucleotide base attached to a marker or tag, wherein the marker or tag comprises a specific moiety having a unique affinity for a ligand. Alternatively, a binding partner may have affinity for the marker or tag. In some examples, the marker includes, but is not limited to, a biotin, a histidine marker (i.e., 6×His), or a FLAG marker. For example, dATP-Biotin may be considered a labeled nucleotide. In some examples, a fragmented nucleic acid sequence may undergo blunting with a labeled nucleotide followed by blunt-end ligation. The term “label” or “detectable label” are used herein, to refer to any composition detectable by spectroscopic, photochemical, biochemical, immunochemical, electrical, optical or chemical means. Such labels include biotin for staining with labeled streptavidin conjugate, magnetic beads (e.g., Dynabeads™), fluorescent dyes (e.g., fluorescein, Texas red, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. The labels contemplated in the present invention may be detected or isolated by many methods.

“Affinity binding molecules” or “specific binding pair” herein means two molecules that have affinity for and bind to each other under certain conditions, referred to as binding conditions. Biotins and streptavidins (or avidins) are examples of a “specific binding pair,” but the invention is not limited to use of this particular specific binding pair. In many embodiments of the present invention, one member of a particular specific binding pair is referred to as the “affinity tag molecule” or the “affinity tag” and the other as the “affinity-tag-binding molecule” or the “affinity tag binding molecule.” A wide variety of other specific binding pairs or affinity binding molecules, including both affinity tag molecules and affinity-tag-binding molecules, are known in the art (e.g., see U.S. Pat. No. 6,562,575) and can be used in the present invention. For example, an antigen and an antibody, including a monoclonal antibody, that binds the antigen is a specific binding pair. Also, an antibody and an antibody binding protein, such as Staphylococcus aureus Protein A, can be employed as a specific binding pair. Other examples of specific binding pairs include, but are not limited to, a carbohydrate moiety which is bound specifically by a lectin and the lectin; a hormone and a receptor for the hormone; and an enzyme and an inhibitor of the enzyme.

As used herein, the term “oligonucleotide” refers to a short polynucleotide, typically less than or equal to 300 nucleotides long (e.g., in the range of 5 and 150, preferably in the range of 10 to 100, more preferably in the range of 15 to 50 nucleotides in length). However, as used herein, the term is also intended to encompass longer or shorter polynucleotide chains. An “oligonucleotide” may hybridize to other polynucleotides, therefore serving as a probe for polynucleotide detection, or a primer for polynucleotide chain extension.

“Extension nucleotides” refer to any nucleotide capable of being incorporated into an extension product during amplification, i.e., DNA, RNA, or a derivative if DNA or RNA, which may include a label.

The term “chromosome” as used herein, refers to a naturally occurring nucleic acid sequence comprising a series of functional regions termed genes that usually encode proteins. Other functional regions may include microRNAs or long noncoding RNAs, or other regulatory elements. These proteins may have a biological function or they directly interact with the same or other chromosomes (i.e., for example, regulatory chromosomes).

The term “genome” refers to any set of chromosomes with the genes they contain. For example, a genome may include, but is not limited to, eukaryotic genomes and prokaryotic genomes. The term “genomic region” or “region” refers to any defined length of a genome and/or chromosome. Alternatively, a genomic region may refer to a complete chromosome or a partial chromosome. Further, a genomic region may refer to a specific nucleic acid sequence on a chromosome (i.e., for example, an open reading frame and/or a regulatory gene).

The term “fragments” refers to any nucleic acid sequence that is shorter than the sequence from which it is derived. Fragments can be of any size, ranging from several megabases and/or kilobases to only a few nucleotides long. Experimental conditions can determine an expected fragment size, including but not limited to, restriction enzyme digestion, sonication, acid incubation, base incubation, microfluidization etc.

The term “fragmenting” refers to any process or method by which a compound or composition is separated into smaller units. For example, the separation may include, but is not limited to, enzymatic cleavage (i.e., for example, transposase-mediated fragmentation, restriction enzymes acting upon nucleic acids or protease enzymes acting on proteins), base hydrolysis, acid hydrolysis, or heat-induced thermal destabilization.

The term “fixing,” “fixation” or “fixed” refers to any method or process that immobilizes any and all cellular processes. A fixed cell, therefore, accurately maintains the spatial relationships between intracellular components at the time of fixation. Many chemicals are capable of providing fixation, including but not limited to, formaldehyde, formalin, or glutaraldehyde.

The term “crosslinking” or “crosslink” refers to any stable chemical association between two compounds, such that they may be further processed as a unit. Such stability may be based upon covalent and/or non-covalent bonding. For example, nucleic acids and/or proteins may be cross-linked by chemical agents (i.e., for example, a fixative) such that they maintain their spatial relationships during routine laboratory procedures (i.e., for example, extracting, washing, centrifugation etc.)

The term “ligated” as used herein, refers to any linkage of two nucleic acid sequences usually comprising a phosphodiester bond. The linkage is normally facilitated by the presence of a catalytic enzyme (i.e., for example, a ligase) in the presence of co-factor reagents and an energy source (i.e., for example, adenosine triphosphate (ATP)).

The term “restriction enzyme” refers to any protein that cleaves nucleic acid at a specific base pair sequence.

As used herein, the term “hybridization” refers to the pairing of complementary (including partially complementary) polynucleotide strands. Hybridization and the strength of hybridization (e.g., the strength of the association between polynucleotide strands) is impacted by many factors well known in the art including the degree of complementarity between the polynucleotides, stringency of the conditions involved affected by such conditions as the concentration of salts, the melting temperature (Tm) of the formed hybrid, the presence of other components, the molarity of the hybridizing strands and the G:C content of the polynucleotide strands. When one polynucleotide is said to “hybridize” to another polynucleotide, it means that there is some complementarity between the two polynucleotides or that the two polynucleotides form a hybrid under high stringency conditions. When one polynucleotide is said to not hybridize to another polynucleotide, it means that there is no sequence complementarity between the two polynucleotides or that no hybrid forms between the two polynucleotides at a high stringency condition.

In one embodiment, a highly sensitive and cost-effective method for genome-wide identification of chromatin interactions in eukaryotic cells is provided. Combining proximity ligation with chromatin immunoprecipitation and sequencing, this method exhibits superior sensitivity, accuracy and ease of operation. For example, application of the method to eukaryotic cells improves mapping of enhancer-promoter interactions.

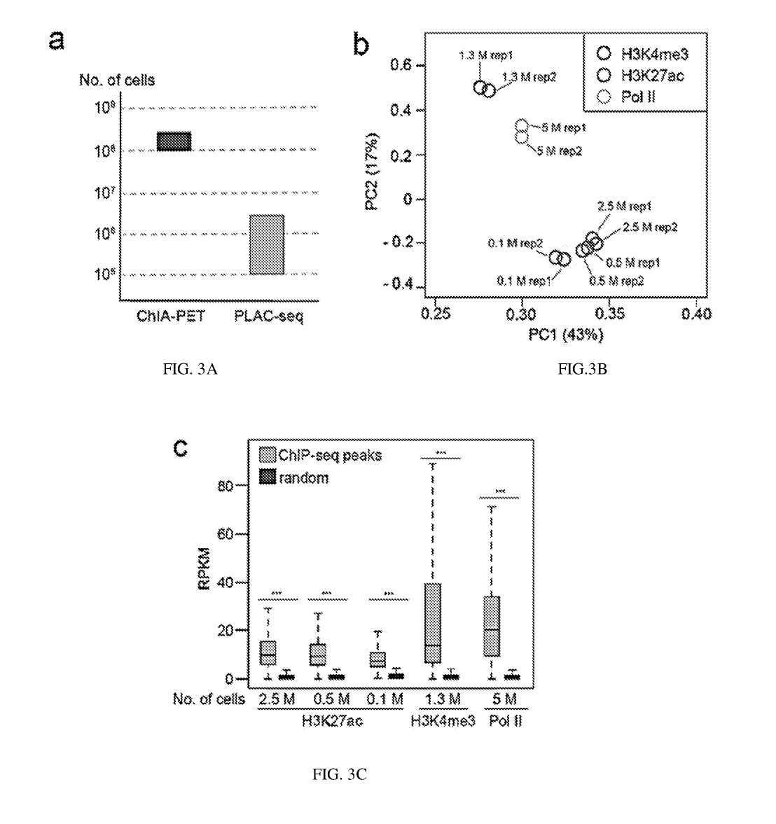

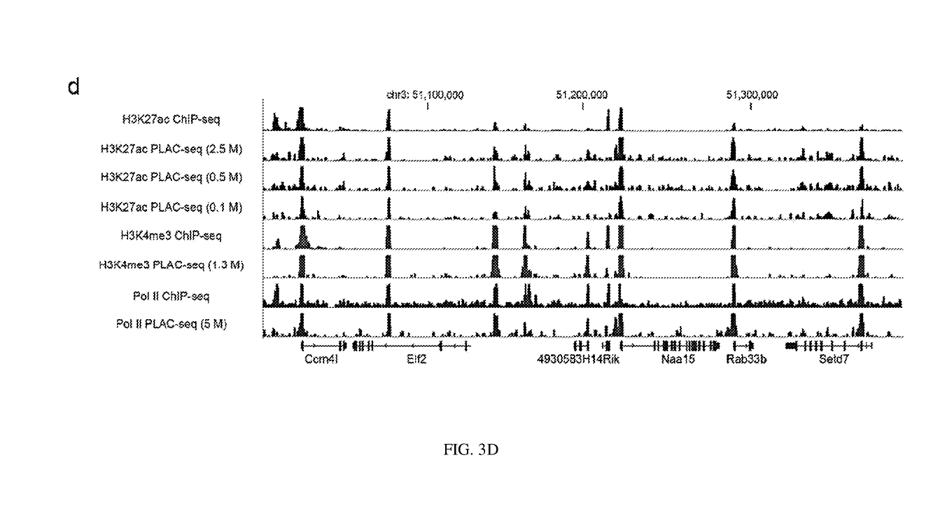

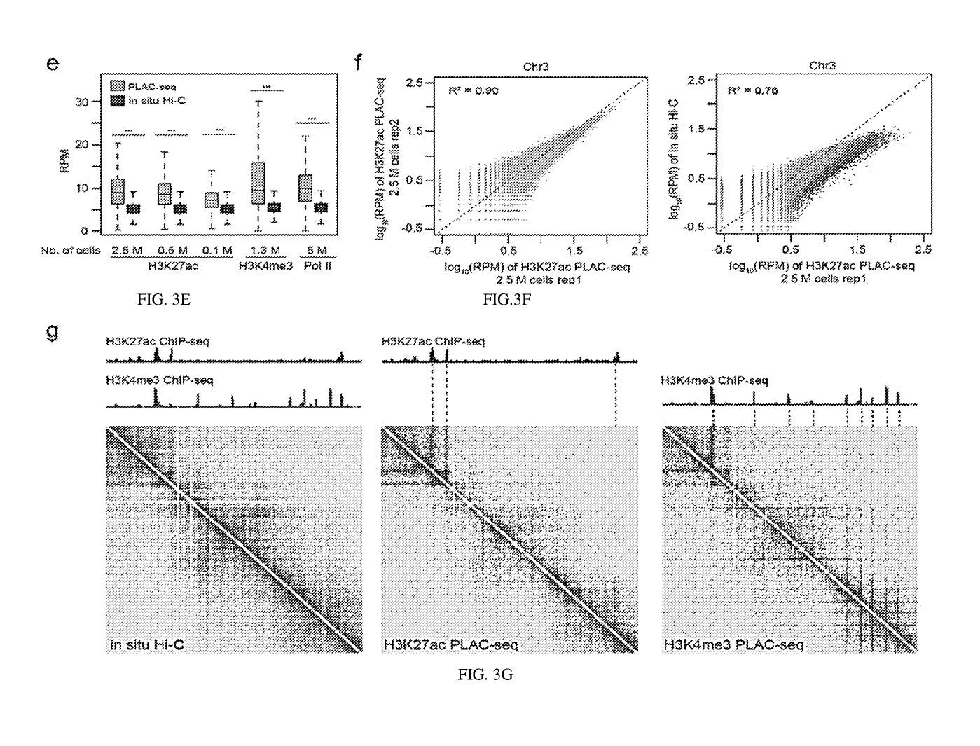

To reduce the amount of input material without compromising the robustness of long-range chromatin interaction mapping, in one embodiment, a method referred to herein as Proximity Ligation Assisted ChIP-seq (PLAC-seq) is provided, which combines formaldehyde crosslinking and in situ proximity ligation with chromatin immunoprecipitation and sequencing (FIG. 1a). PLAC-seq can detect long-range chromatin interactions in a more comprehensive and accurate manner while using as few as 100,000 cells, or three orders of magnitude less than published ChIA-PET protocols (Fullwood, M. J. et al., Nature 462, 58-64 (2009) and Tang, Z. et al., Cell 163, 1611-1627 (2015)) (FIG. 3a). In one embodiment, PLAC-seq was performed with mouse ES cells and using antibodies against RNA Polymerase II (Pol II), H3K4me3 and H3K37ac to determine long-range chromatin interactions at genomic locations associated with the transcription factor or chromatin marks (Table 1).

The complexity of the sequencing library generated from PLAC-seq is much higher than ChIA-PET when comparing the Pol II PLAC-seq and ChIA-PET experiments. As a result, 10× more sequence reads were obtained 440 times more monoclonal cis long-range (>10 kb) read pairs were collected from a Pol II PLAC-seq experiment than a previously published Pol II ChIA-PET experiment (Zhang, Y. et al., Nature 504, 306-310 (2013)) (FIG. 1b). In addition, PLAC-seq library has substantially fewer inter-chromosomal pairs (11% vs. 48%), but much more long-range intra-chromosomal pairs (67% vs. 9%) and significantly more usable reads for interaction detection (25% vs. 0.6%). Therefore, PLAC-seq is much more cost-effective than ChIA-PET (FIG. 1b).

TABLE 1

| | | | | cis pairs within 500 | | |
|---|---|---|---|---|---|---|
| Number of cell | | Uniquely mapped pairs | | bp of Mbol cutting | long-range (>10 kb) | unique long- |
| used (million) | ChIP Antibody | (qual > 10) | cis pairs | sites | cis pairs | range cis pairs |
| 2.5M (replicate 1) | H3K27ac | 131,187,822 | 120,500,656 | 118,668,487 | 71,200,523 | 61,477,778 |
| 2.5M (replicate 2) | H3K27ac | 139,664,576 | 128,504,835 | 126,786,302 | 74,578,145 | 64,791,520 |
| 0.5M (replicate 1) | H3K27ac | 110,351,215 | 100,252,104 | 99,087,234 | 62,605,541 | 51,441,531 |
| 0.5M (replicate 2) | H3K27ac | 102,218,352 | 93,165,698 | 92,245,938 | 57,100,632 | 47,145,994 |
| 1.3M (replicate 1) | H3K4me3 | 121,570,664 | 110,681,678 | 109,362,518 | 64,632,025 | 54,762,522 |
| 1.3M (replicate 2) | H3K4me3 | 115,470,150 | 104,808,865 | 103,417,392 | 59,337,747 | 49,720,878 |
| 5M (replicate 1) | Pol II | 107,268,403 | 95,917,316 | 94,371,244 | 63,293,924 | 44,040,125 |
| 5M (replicate 2) | Pol II | 92,897,183 | 82,410,294 | 80,664,861 | 52,291,140 | 30,269,147 |

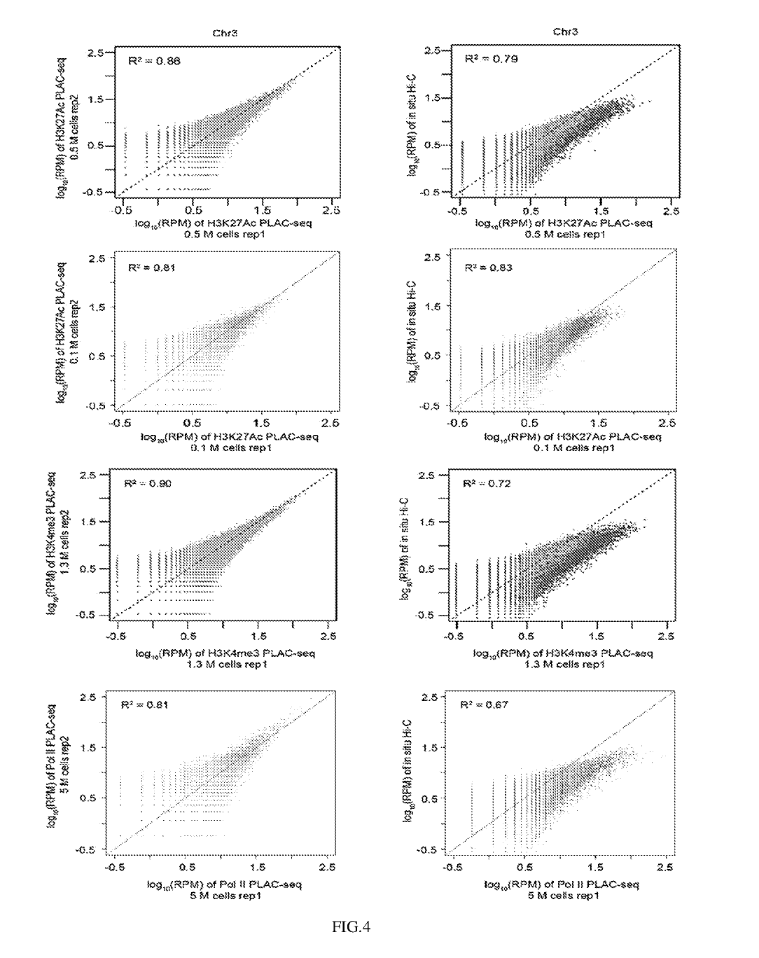

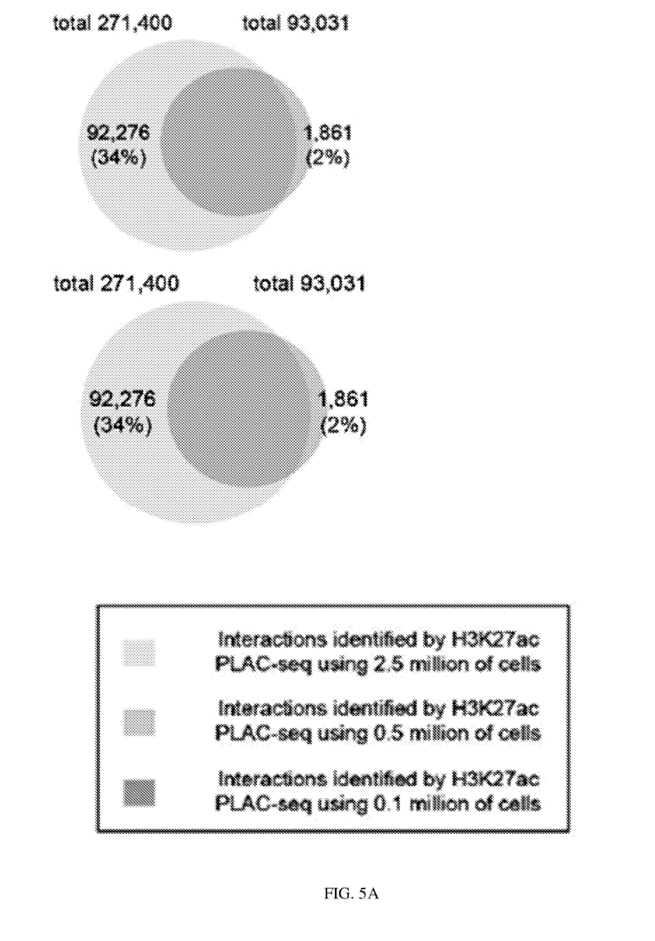

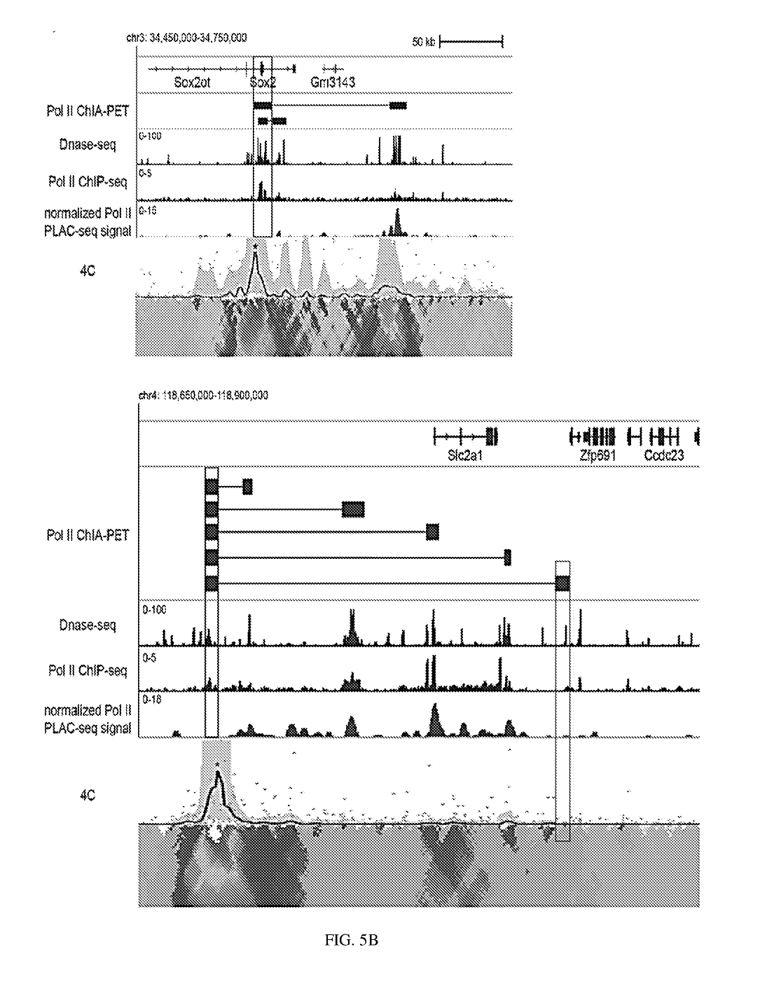

To evaluate the quality of PLAC-seq data, it was first compared with the corresponding ChIP-seq data previously collected for mouse ES cells (ENCODE) (Shen, Y. et al., Nature 488, 116-120 (2012)) and it was found that PLAC-seq reads were significantly enriched in factor binding sites (P<2.2e-16) and are highly reproducible between biological replicates (Pearson correlation >0.90) (FIG. 3b-g, FIG. 4). Therefore, the data from two biological replicates were combined for subsequent analysis. A published algorithm ‘GOTHiC’ (Schoenfelder, S. et al., Genome Res. 25, 582-597 (2015)) was used to identify long-range chromatin interactions in each dataset. Highly reproducible interactions identified by H3K27ac PLAC-seq using 2.5, 0.5 and 0.1 million of cells were observed (FIG. 5a). Furthermore, PLAC-seq signals normalized by in situ Hi-C data revealed interactions at sub-kilobasepair resolution even with 100,000 cells (FIG. 1c-d). A total of 60,718, 271,381, and 188,795 significant long-range interactions were identified from Pol II, H3K27ac or H3K4me3 PLAC-seq experiment, respectively.

Previously, ChIA-PET was performed for Pol II in mouse ES cells, providing a reference dataset for comparison (Zhang, Y. et al., Nature 504, 306-310 (2013)). After examining the raw read counts from the PLAC-seq interacting regions, it was found that each chromatin contact was typically supported by 20 to 60 unique reads. By contrast, chromatin interactions identified in ChIA-PET analysis were generally supported by fewer than 10 unique pairs (Zhang, Y. et al., Nature 504, 306-310 (2013)) (FIG. 1e). Next, it was found that Pol II PLAC-seq analysis identified a lot more interactions than Pol II ChIA-PET (˜60,000 vs. ˜10,000), with 10% PLAC-seq overlapping with 35% of ChIA-PET intra-chromosomal interactions (FDR <0.05 and PET count >=3) (FIG. 1f). To further investigate the sensitivity and accuracy of each method, in situ Hi-C was performed on the same cell line and 300 million unique long-range (>10 kb) cis pairs were collected from 93-1.2 billion paired-end sequencing reads. Using ‘GOTHiC’, 464,690 long-range chromatin interactions were identified. It was found that 94% of the chromatin interactions found in Pol II PLAC-seq overlapped with 28% of in situ Hi-C interactions, while 44% of contacts detected by ChIA-PET matched less than 2% of that of in situ Hi-C contacts (FIG. 1g). The H3K27ac and H3K4me3 PLAC-seq interactions were also examined and it was found that the interactions identified by these two marks together recovered 68% of the in situ Hi-C interactions (FIG. 1h). In addition, it was observed that PLAC-seq interactions in general have a higher coverage on regulatory elements such as promoters and distal DNase I hypersensitive sites (DHSs) compared to ChIA-PET (FIG. 1i). Taken together, the disclosure above supports the superior sensitivity and specificity of PLAC-seq over ChIA-PET.

To further validate the reliability of PLAC-seq, 4C-seq analysis was performed at four selected regions (Table 2).

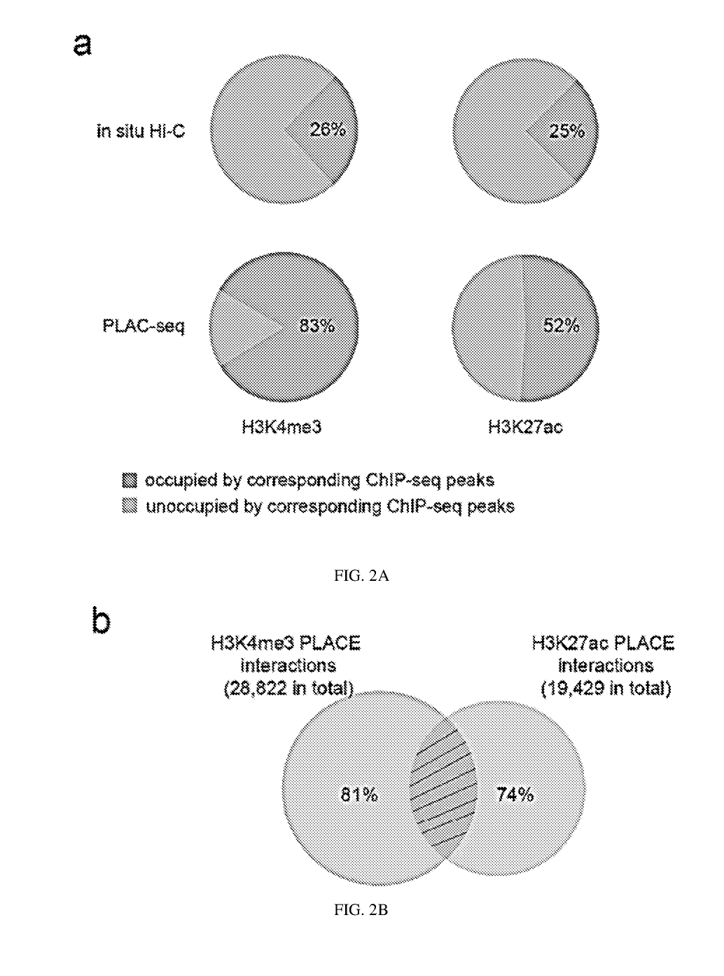

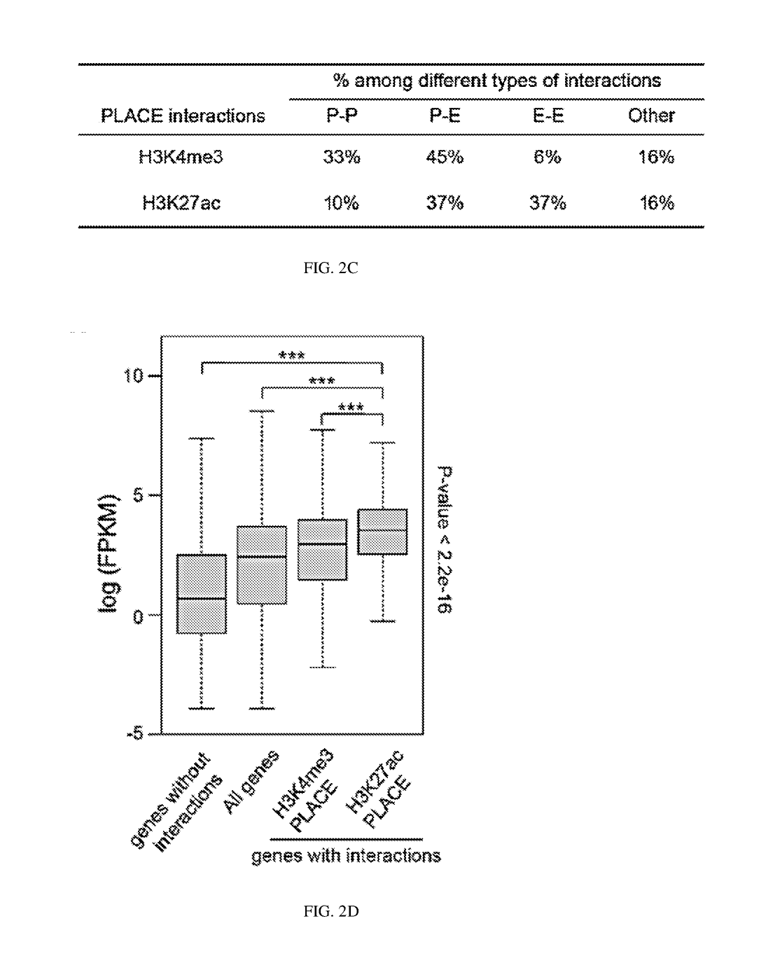

Although most interactions were independently detected by both ChIA-PET and PLAC-seq methods (FIG. 1j, left panel, and FIG. 5b), there were three strong interactions (marked 1,2,3 in FIG. 1j) determined by 4C-seq that were detected by PLAC-seq, but not ChIA-PET. Conversely there was a case of chromatin interaction uniquely detected by ChIA-PET but not observed from 4C-seq (highlighted by the right rectangle in FIG. 5b), once again supporting the superior performance of PLAC-seq over ChIA-PET. H3K4me3 and H3K27ac PLAC-seq datasets were examined to study promoter and active enhancer interactions in the mouse ES cells. PLAC-seq interactions were highly enriched with the corresponding ChIP-seq peaks compared to in situ Hi-C interactions (FIG. 2a). The enrichment allowed further exploration of interactions specifically enriched in PLAC-seq compared to in situ Hi-C due to chromatin immunoprecipitation. Identifying such interactions allows understanding of higher-order chromatin structures associated with a specific protein or histone mark. To achieve this, a computational method was developed using Binomial test to detect interactions that are significantly enriched in PLAC-seq relative to in situ Hi-C. This type of interactions was termed as ‘PLACE’ (PLAC-Enriched) interactions. A total of 28,822 and 19,429 significant H3K4me3 or H3K27ac PLACE interactions (q<0.05) (FIG. 4,5) in the mouse ES cells were identified, respectively. 26% of H3K27ac PLACE interactions overlapped with 19% of H3K4me3 PLACE interactions, indicating that they contain different sets of chromatin interactions (FIG. 2b). The majority of H3K27ac PLACE interactions are enhancer-associated interactions (74%) while H3K4me3 PLACE interactions are generally associated with promoters (78%) (FIG. 2c). The difference between H3K27ac and H3K4me3 PLACE interactions led to further investigation of these two types of interactions. The expression levels of genes associated with H3K27ac and H3K4me3 PLACE interactions was examined and it was determined that genes involved in H3K27ac PLACE interactions have a significantly higher expression level than genes associated with H3K4me3 PLACEinteractions (P<2.2e-16, FIG. 2d), indicating that the former assay is useful to discover chromatin interactions at active enhancers.

TABLE 2

| | | 1st | 2nd | | | |
|---|---|---|---|---|---|---|
| Sample | | digestion | digestion | | | FIG. |
| No. | Anchor point | enzyme | enzyme | PCR primer (forward) | PCR primer (reverse) | related |
| 4C_1 | Chr: | Csp6I | NlaIII | TCCCTACACGACGCTCTTCCGAT | GTGACTGGAGTTCAGACGTGTGC | FIG. 5b, |
| | 34,545,849- | | | CTATTGCCTCTGATAAGTAC | TCTTCCGATCTATGACAGCCCCA | upper |
| | 34,546,065 | | | (SEQ ID NO: 1) | GCCCAT | panel |
| | | | | | (SEQ ID NO: 2) | |
| 4C_2 | Chr1: | DpnII | Csp6I | TCCCTACACGACGCTCTTCCGAT | GTGACTGGAGTTCAGACGTGTGC | FIG. 1J, |
| | 72,261,052- | | | CTAGACAAGCCTCAGTTGGATC | TCTTCCGATCTATCCCAAGGCTA | left |
| | 72,261,738 | | | (SEQ ID NO: 3) | CATCATTA | |
| | | | | | (SEQ ID NO: 4) | |
| 4C_3 | Chr5: | DpnII | Csp6I | TCCCTACACGACGCTCTTCCGAT | GTGACTGGAGTTCAGACGTGTGC | FIG. 1J, |
| | 110,901,207- | | | CTGGGAGTCATGGAAACTGATC | TCTTCCGATCTTTGATAGTAACA | right |
| | 110,901-593 | | | (SEQ ID NO: 5) | AGGCCCC | |
| | | | | | (SEQ ID NO: 6) | |
| 4C_4 | Chr4: | DpnII | Csp6I | TCCCTACACGACGCTCTTCCGAT | GTGACTGGAGTTCAGACGTGTGC | FIG. 5b, |
| | 118,684,035- | | | CTATTCTTCTTCTGAAAGGATC | TCTTCCGATCTATTTTAGCGGAA | lower |
| | 118,684,927 | | | (SEQ ID NO: 7) | GACTCACA | panel |
| | | | | | (SEQ ID NO: 8) | |

### Examples

Materials and Methods

Cell Culture and Fixation.

The F1 Mus musculus castaneus×S129/SvJae mouse ESC line (F123 line) was a gift from the laboratory of Dr. Rudolf Jaenisch and was previously described in Gribnau, J., et al., Genes & development 17, 759-773 (2003). F123 cells were cultured as described previously in Selvaraj, S. et al., Nat. Biotechnol. 31, 1111-1118 (2013). Cells were passaged once on 0.1% gelatin-coated feeder-free plates before fixation.

To fix the cells, cells were harvested after accutase treatment and suspended in medium without Knockout Serum Replacement at a concentration of 1×10⁶ cells per 1 ml. Methanol-free formaldehyde solution was added to the final concentration of 1% (v/v) and rotated at room temperature for 15 min. The reaction was quenched by addition of 2.5 M glycine solution to the final concentration of 0.2 M with rotation at room temperature for 5 min. Cells were pelleted by centrifugation at 3,000 rpm for 5 min at 4° C. and washed with cold PBS once. The washed cells were pelleted again by centrifugation, snap-frozen in liquid nitrogen and stored at −80° C.

PLAC-Seq Protocol.

PLAC-seq protocol contains three parts: in situ proximity ligation, chromatin immunoprecipitation or ChIP, biotin pull-down followed by library construction and sequencing. The in situ proximity ligation and biotin pull-down procedures were similar to previously published in situ Hi-C protocol (Rao, S. S. P. et al., Cell 159, 1665-1680 (2014)) with minor modifications as described below:

1. In situ proximity ligation. 0.5 to 5 million of crosslinked F123 cells were thawed on ice, lysed in cold lysis buffer (10 mM Tris, pH 8.0, 10 mM NaCl, 0.2% IGEPAL CA-630 with proteinase inhibitor) for 15 min, followed by a washing step with lysis buffer once. Cells were then resuspended in 50 μl 0.5% of SDS and incubated at 62° C. for 10 min. Permeabilization was quenched by adding 25 μl 10% Triton X-281100 and 145 μl water, and incubation at 37° C. for 15 min. After adding NEBuffer 2 to 1× and 100 units of MboI, the digestion was performed for 2 h 37° C. in a thermomixer, shaking at 1,000 rpm. After inactivation of MboI at 62° C. for 20 min, biotin fill-in reaction was performed for 1.5 h 37° C. in a thermomixer after adding 15 nmol of dCTP, dGTP, dTTP, biotin-14-dATP (Thermo Fisher Scientific) each and 40 unit of Klenow. Proximity ligation was performed at room temperature with slow rotation in a total volume of 1.2 ml containing 1×T4 ligase buffer, 0.1 mg/ml BSA, 1% Triton X-100 and 4000 unit of T4 ligase (NEB).

2. ChlIP. After proximity ligation, the nuclei were spun down at 2,500 g for 5 min and the supernatant was discarded. The nuclei were then resuspended in 130 μl RIPA buffer (10 mM Tris, pH 8.0, 140 mM NaCl, 1 mM EDTA, 1% Triton X-100, 0.1% SDS, 0.1% sodium deoxycholate) with proteinase inhibitors. The nuclei were lysed on ice for 10 min and then sonicated using Covaris M220 with following setting: power, 75 W; duty factor, 10%; cycle per burst, 200; time, 10 min; temp, 7° C. After sonication, the samples were cleared by centrifugation at 14,000 rpm for 20 min and supernatant was collected. The clear cell lysate was mixed with Protein G Sepharose beads (GE Healthcare) and then rotated at 4° C. for pre-clearing. After 3 h, supernatant was collected and ˜5% of lysate was saved as input control. The rest of the lysate was mixed with 2.5 μg of H3K27Ac (ab4729, ABCAM), H3K4me3 (04-745, MILLIPORE) or 5 μg Pol II (ab817, ABCAM) specific antibody and incubate at 4° C. overnight. On the next day, 0.5% BSA-blocked Protein G Sepharose beads (prepared one day ahead) were added and rotated for another 3 h at 4° C. The beads were collected by centrifugation at 2,000 rpm for 1 min and then washed with RIPA buffer three times, high-salt RIPA buffer (10 mM Tris, pH 8.0, 300 mM NaCl, 1 mM 1 EDTA, 1% Triton X-100, 0.1% SDS, 0.1% sodium deoxycholate) twice, LiCl buffer (10 mM Tris, pH 8.0, 250 mM LiCl, 1 mM EDTA, 0.5% IGEPAL CA-630, 0.1% sodium deoxycholate) once, TE buffer (10 mM Tris, pH 8.0, 0.1 mM EDTA) twice. Washed beads were first treated with 10 μg Rnase A in extraction buffer (10 mM Tris, pH 8.0, 350 mM NaCl, 0.1 mM EDTA, 1% SDS) for 1 h at 37° C. Then 20 μg proteinase K was added and reverse crosslinking was performed overnight at 65° C. The fragmented DNA was purified by Phenol/Chloroform/Isoamyl Alcohol (25:24:1) extraction and ethanol precipitation.

3. Biotin pull-down and library construction. The biotin pull-down was performed according to in situ Hi-C protocol with the following modifications: 1) 20 μl of Dynabeads MyOne Streptavidin T1 beads were used per sample instead of 150 μl per sample; 2) To maximize the PLAC-seq library complexity, the minimal number of PCR cycles for library amplification was determined by qPCR.

PLAC-Seq and Hi-C Read Mapping.

A bioinformatics pipeline was developed to map PLAC-seq and in-situ Hi-C data. Paired-end sequences were first mapped using BWA-MEM (Li H. Aligning sequence reads, clone sequences and assembly contigs with BWA-MEM. arXiv:1303.3997v2 (2013)) to the reference genome (mm9) in single-end mode with default setting for each of the two ends separately. Next, independently mapped ends were paired up and pairs were only kept if each of both ends were uniquely mapped (MQAL>10). As the focus was on intrachromosomal analysis in this study, interchromosomal pairs were discarded. Next, read pairs were further discarded if either end was mapped more than 500 bp apart away from the closest MboI site. Read pairs were next sorted based on genomic coordinates followed by PCR duplicate removal using MarkDuplicates in Picard tools. Finally, the mapped pairs were partitioned into “long-range” and “short-range” if its insert size was greater than the given distance of default threshold 10 kb or smaller than 1 kb, respectively.

PLAC-Seq Visualization.

For each given anchor point, the interaction read pairs with one end falling in the anchor region, the other flanking outside it, were first extracted. Next, the 2 MB window surrounding the anchor point was split into a set of 500 bp non-overlapping bins. The flanking read was extended into 2 kb, then the coverage for each bin from both PLAC-seq and in situ Hi-C experiments was counted. The read count was later normalized into RPM (Read Per Million) and the final normalized PLAC-seq signal was the subtraction between treatment and input.

PLAC-Seq and In Situ Hi-C Interaction Identification.

‘GOTHiC’ (Schoenfelder, S. et al., Genome Res. 25, 582-597 (2015)) was used to identify long-range chromatin interactions in PLAC-seq and in situ Hi-C datasets with 5 kb resolution. To identify the most convincing interactions, an interaction was considered significant if its FDR <1e-20 and read count >20. In total, 60,718, 271,381, 188,795 significant long-range interactions were identified from Pol II, H3K27ac, H3K4me3 PLAC-seq and 464,690 from in situ Hi-C in the mouse ES cells.

Interaction Overlap.

Two distinct interactions are defined as overlapped if both ends of each interaction intersect by at least one base pair.

Identification of PLACE Interactions.

H3K4me3/H3K27ac/Pol2 ChIP-seq peaks in mouse ES cells were downloaded from ENCODE (Shen, Y. et al., Nature 488, 116-120 (2012)). Each peak was expanded to 5 kb as an anchor point. PLAC-Enriched (PLACE) interactions were identified by the exact binomial test using in situ Hi-C as an estimation of background interaction frequency. In greater detail, for each anchor region i, the number of read pairs having one end overlap with anchor region read_total_treatᵢ and read_total_inputᵢ for PLAC-seq and in situ Hi-C were first counted. Next, the focus was on a 2 MB window flanking the anchor and partitioned this region into a set of overlapping 5 kb bins with a step size of 2.5 kb. Briefly, the probability that a read pair is the result of a spurious ligation between the anchor region i and bin j can be estimated as:

Pᵢⱼ=inputᵢⱼ/total_inputᵢ

Then, the probability of observing treaty read-pairs in PLAC-seq between i and bin j can be calculated by the binomial density:

$${pval}_{i , j} = P ( x > {treat}_{ij} ) = 1 - {\sum }_{m = 0}^{{treat}_{ij}} ( \begin{matrix}{total\_treat}_{i} \\ m\end{matrix} ) {( {P}_{ij} )}^{m} {( 1 - {P}_{ij} )}^{( {total\_treat}_{i} - m )}$$

Next, bins that have a binomial P value smaller than 1e-5 were identified as candidates. Centering on each candidate, a 1 kb, 2 kb, 3 kb, 4 kb window was chosen and the fold change calculated respectively, then the peak with the largest fold change was defined as an interaction:

Fₘₐₓ=max(F_{1K},F_{2K},F_{3K},F_{4K})

Overlapping interactions were merged as one interaction and binomial P was recalculated based on the merged interaction. Next, the resulting P values were corrected to q value to account for multiple hypothesis testing using Bonferroni correction. Finally, interactions with q value smaller than 0.05 were reported as significant interactions.

Hi-C and PLAC-Seq Contact Maps Visualization.

In situ Hi-C or PLAC-seq contact maps were visualized using Juicebox (Durand, N. C. et al., Cell Systems 3, 99-101 (2016)) after removing all trans reads and cis reads pairs span less than 10 kb.

4C Validation.

4C experiments were performed as previously described in van de Werken, H. J. G. et al. in Nucleosomes, Histones & Chromatin Part B 513, 89-112 (Elsevier, 2012). The restriction enzymes used and the primer sequences for PCR amplification are listed in Table 2. Data analysis was performed using 4Cseqpipe in the manner described in van de Werken, H. J. G. et al., Nat. Methods 9, 969-972 (2012).

In Situ Hi-C.

F123 in situ Hi-C was performed as previously described in Rao, S. S. P. et al., Cell 159, 1665-1680 (2014) with 5 million of F123 cells.

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims. All references cited herein are incorporated by reference herein in their entireties.

1. A method for genome-wide identification of chromatin interactions in a cell comprising: providing a cell that contains a set of chromosomes having genomic DNA;
incubating the cell or the nucleus thereof with a fixation agent to provide a fixed cell comprising a complex having genomic DNA crosslinked with a protein;
performing proximity ligation of the genomic DNA of the fixed cell to form proximally-ligated genomic DNA;
isolating the complex from the cell to provide a DNA library; and
sequencing the DNA library.

2. The method of claim 1, further comprising shearing the proximally-ligated genomic DNA before the isolating step.

3. The method of claim 2, wherein the shearing is carried out by sonication.

4. The method of claim 1 wherein the fixation agent is formaldehyde, glutaraldehyde, formalin, or a mixture thereof.

5. The method of claim 1 wherein the proximity ligation is an in situ ligation performed by a process comprising permeabilizing the fixed cell;
fragmenting the genomic DNA, and
performing labeled nucleotide fill-in with a labeled nucleotide and
ligating the genomic DNA to form proximally-ligated genomic DNA.

6. The method of claim 1 wherein the cell containing a set of chromosomes having genomic DNA or the nucleus thereof is lysed before the proximity ligation step.

7. The method of claim 5, wherein fragmenting step is carried out by restriction digestion with an enzyme.

8. The method of claim 7, wherein the enzyme is a 4-cutter or a 6-cutter.

9. The method of claim 5, wherein the labeled nucleotide is labeled with a tag.

10. The method of claim 9, wherein the tag is biotin.

11. The method of claim 1, further comprising pulling down the genomic DNA from the complex after the isolating step and prior to the sequencing step.

12. The method of claim 1, wherein the complex is isolated by immunoprecipitation using an antibody that specifically binds to the protein.

13. The method of claim 12, wherein the protein is a transcription factor.

14. The method of claim 1, wherein the cell is a mammalian cell or derived from a tissue.

15. A kit for performing the method of claim 1, comprising one or more reagents selected from the following: a fixative agent, a restriction endonuclease, a ligase, a DNA-binding protein, a labeled nucleotide, a capturing agent, an antibody or an antigen binding portion thereof, adaptor oligonucleotides and/or sequencing primers, a lysis buffers, dNTPs, a polymerase, a polynucleotide kinase, a ligase buffer, and PCR reagents and a biological sample.

16. The kit of claim 15, wherein the capturing agent is streptavidin.
